(19) United States
(12) Patent Application Publication
(10) Pub. No.: US 2024/0180905 A1
(43) Pub. Date: Jun. 6, 2024

(54) USES OF FATTY ACID AMIDE HYDROLASE INHIBITORS IN TREATMENT OF TRAUMA RELATED PSYCHIATRIC DISORDERS

(71) Applicant: NEURITEK LTD., New York, NY (US)

(72) Inventors: William E. Hapworth, New York, NY (US); Alexander Neumeister, Waltham, MA (US)

(73) Assignee: NEURITEK LTD., New York, NY (US)

(21) Appl. No.: 18/546,753

(22) PCT Filed: Jul. 19, 2021
(86) PCT No.: PCT/US21/42137

§ 371 Date: Aug. 16, 2023

Related U.S. Application Data
(60) Provisional application No. 63/054,483, filed on Jul. 21, 2020.

(51) Int. Cl. A61K 31/501; A61K 45/06; A61P 25/22

(52) U.S. Cl. CPC A61K 31/501; A61K 45/06; A61P 25/22

(57) ABSTRACT

The present invention is directed to compositions containing a fatty acid amide hydrolase inhibitor and methods of use thereof for treatment of trauma-related psychiatric disorders in a patient in need thereof, particularly those that exhibiting hyperarousal symptomatology.

### CROSS-REFERENCE TO RELATED APPLICATION

This application claims priority under 35 U.S.C. § 119(e) to U.S. Provisional Patent Application Ser. No. 63/054,483, filed on Jul. 21, 2020, which is hereby incorporated by reference in its entirety.

### BACKGROUND

Posttraumatic stress disorder (PTSD) is a major public health concern. According to the U.S. population-based studies such as the National Comorbidity Survey (NCS), NCS-Replication, and the National Epidemiologic Survey on Alcohol and Related Conditions, the lifetime prevalence of PTSD ranges from 6.4% to 7.8%. PTSD is a chronic disorder and the majority of individuals with PTSD either receive no or suboptimal treatment.

The current approach for PTSD includes a cognitive-behavioral arm consisting of exposure therapy and relaxation techniques, as well as a pharmacological arm consisting mainly of antidepressant medication such as sertraline (Zoloft®) or paroxetine (Paxil®). Despite availability of a few pharmacological options, many patients receiving these treatments will remain non-responsive.

Moreover, typical pharmacological treatments require daily compliance with medication intake, and there are long-term and potentially disabling side effects associated with long-term use of these medications, which in addition to non-responsiveness negatively impacts compliance. To that end, there is a need in the art to address and provide additional alternative treatments for PTSD. The present invention addresses this need.

### SUMMARY OF THE INVENTION

The present invention addresses the shortcomings in the art. At least one aspect of the present invention concerns methods of treating a patient suffering or at risk of developing a trauma-related psychiatric disorder with a compound that is able to inhibit the activity of fatty acid amide hydrolase (FAAH) in the patient. In some embodiments, the compound is an azetidine derivative. In some embodiments, the present invention is directed to methods of treating a patient who is suffering or is at risk of developing a trauma-related psychiatric disorder by administering therapeutically effective amounts of 3-{[5-(2-Fluorophenyl)pyridin-2-yl]oxy}-N-(pyridazin-3-yl)azetidine-1-carboxamide or a pharmaceutically acceptable salt thereof.

In some embodiments, the compound 3-{[5-(2-Fluorophenyl)pyridin-2-yl]oxy}-N-(pyridazin-3-yl)azetidine-1-carboxamide is in a pharmaceutically acceptable salt or complex providing the desired therapeutic activity. In some embodiments, the 3-{[5-(2-Fluorophenyl)pyridin-2-yl]oxy}-N-(pyridazin-3-yl)azetidine-1-carboxamide is a salt of hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, or polygalacturonic acid.

In some embodiments, the azetidine derivative is administered at doses ranging from 0.1 mg to 1000 mg, preferably ranging from 1 mg to 750 mg. In other embodiments, the azetidine derivative is in the form of an oral immediate, delayed, sustained or controlled release formulation.

At least another aspect of the present invention concerns methods of treating trauma-related psychiatric disorders in a patient in need thereof following the steps of (a) selecting a patient suffering from symptoms associated with said disorder, (b) administering to the patient effective amounts of a fatty acid amide hydrolase (FAAH) inhibitor, (c) identifying an improvement of at least one of the patient's symptoms from the baseline. In some embodiments, the post-traumatic psychiatric disorder is any one of the post-traumatic stress disorder (PTSD), post-traumatic anxiety disorder, post-traumatic depression, Major Depressive Disorder (MDD), substance use disorder, abnormal eating behaviors, and Global Developmental Delay (GDD) or any combinations thereof. In other embodiments, the subject patient with the post-traumatic psychiatric disorder suffers from at least one of the symptoms associated to hyperarousal and exaggerated startle. In other embodiments, the patient's hyperarousal symptoms may be any one of (a) irritable behavior, (b) angry outbursts, (c) recklessness, (d) self-destructive behavior, (e) hypervigilance, (f) exaggerated startle response, (g) problems with concentration; (i) sleep disturbances or any combinations thereof.

In other embodiments, the hyperarousal symptom is manifested for at least one month, two months, three months or more after the patient's exposure to the underlying trauma. In certain embodiments, the trauma is secondary to a fear-conditioning model. In certain embodiments, the patient may also be evaluated for a primary somatic root cause of the phenotype.

In some embodiments, the patient suffering from or at risk of developing trauma-related psychiatric disorders may further exhibit abnormal cortisol levels, abnormal endocannabinoid concentrations, a smaller hippocampus than average population measurements, or any combinations thereof.

In another aspect of the present invention, the patient is treated with a combination of therapeutically effective amount of an azetidine derivative and a second therapeutic agent such as a serotonin reuptake inhibitors (SSRIs), a beta-adrenergic antagonist, an alpha 1 adrenergic antagonist, a benzodiazepine, an opiate compound, a cannabinoid compound, a cortisol lowering agent, an antagonist of N-methyl-D-aspartate (NMDA) receptors, a ventromedial prefrontal cortex enhancer and any combinations thereof administered concomitantly or sequentially. In other embodiments, the azetidine derivative is -{[5-(2-Fluorophenyl)pyridin-2-yl]oxy}-N-(pyridazin-3-yl)azetidine-1-carboxamide or a pharmaceutically acceptable salt or complex thereof.

In some embodiments, the present invention is directed to methods of patients at risk of developing post-traumatic psychiatric disorder comprising the steps of (1) determining the patient's endophenotype, (2) among those identified in step (1), selecting those patients who exhibit at least one of the symptom selected from the group consisting of a) irritable behavior, (b) angry outbursts, (c) recklessness, (d) self-destructive behavior, (e) hypervigilance, (f) exaggerated startle response, (g) problems with concentration, (i) sleep disturbances or any combinations thereof, (3) administering to the patients identified in step (2) an effective amount of an FAAH inhibitor, (4) evaluating the patient's improvement of at least one the symptoms, and (5) continue administering the FAAH inhibitor to those patients who show improvement in at least one of the symptoms. In other embodiments, post-traumatic psychiatric disorder is post-traumatic stress disorder (PTSD), post-traumatic anxiety disorder, post-traumatic substance use disorder, post-traumatic eating disorder or post-traumatic depression.

In other embodiments, the patient may further suffer from a symptom that is to hyperarousal and exaggerated startle.

In some embodiments, the patient may further experience symptoms associated with hyperarousal such as (a) irritable behavior, (b) angry outbursts, (c) recklessness, (d) self-destructive behavior, (e) hypervigilance, (f) exaggerated startle response, (g) problems with concentration, (i) sleep disturbances or any combinations thereof. In other embodiments, the hyperarousal symptom is manifested for at least one month after the patient's exposure to trauma. In some embodiments, the trauma is secondary to a fear-conditioning model.

In other embodiments, he patient further exhibits abnormal cortisol levels, abnormal endocannabinoid concentrations, a smaller hippocampus than average population measurements, or a reduced volume of prefrontal regions of the brain as compared to population measurements, or any combinations thereof.

In another aspect, the present invention is directed to a method of treating anxiety, pain associated with depression or anxiety, substance abuse and dependence associated with trauma, abnormal eating behavior associated with trauma, depression associated with trauma following the steps of administering to a subject in need of such treatment an effective amount of a FAAH inhibitor. In some embodiments, the FAAH inhibitor is selected from the group consisting of an azetidine derivative such as 3-{[5-(2-Fluorophenyl)pyridin-2-yl]oxy}-N-(pyridazin-3-yl)azetidine-1-carboxamide or a pharmaceutically acceptable salt thereof. In other embodiments, the azetidine derivative may be administered with a second therapeutic agent, concomitantly or sequentially.

In another aspect, the present invention is directed to a pharmaceutical unit form which comprises an immediate release and a delayed release or controlled release component, wherein the immediate release component comprising a first therapeutic agent selected from the group consisting of a FAAH inhibitor, and the delayed release component comprising a second therapeutic agent that is selected from the group consisting of a serotonin reuptake inhibitors (SSRIs), a beta-adrenergic antagonist, an alpha 1 adrenergic antagonist, a benzodiazepine, an opiate compound, a cannabinoid compound, a cortisol lowering agent, an antagonist of N-methyl-D-aspartate (NMDA) receptors, a ventromedial prefrontal cortex enhancer and any combinations thereof.

At least another aspect of the present invention is to employ optimal characterization of the phenotypic expression of trauma-related psychopathology in factor analytic and taxometric studies as endpoints in clinical trials of pharmacotherapies for trauma-related psychopathology.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 illustrates the brain circuitry that is routinely involved in PTSD and the location of CB1 receptor in different regions of the brain.

FIG. 2 illustrates the relationship between measures of startle response to unpredictable threat in a fear-potentiated startle paradigm and the phenotypic expression of PTSD symptomatology using a novel 5-factor model of this disorder.

FIG. 3 illustrates evidence to implicate impaired CB1 receptor-mediated anandamide (AEA) signaling in the pathogenesis of PTSD. The evidence shows mean plasma AEA levels are significantly lower in PTSD patients (0.72±0.12 pmol/ml) relative to healthy control subjects without trauma history (HC; 2.74±0.85 pmol/ml, t=2.47, df=17, p<0.05 as derived from an unpaired 2-sample t test).

### DETAILED DESCRIPTION OF THE INVENTION

The following description is provided to enable any person skilled in the art to make and use the subject matter of this disclosure, in best modes contemplated for carrying out the various aspects of the disclosure. Various modifications, however, will remain readily apparent to those skilled in the art, since the generic principles of the disclosed subject matter have been defined herein specifically to describe: (1) a method of treating trauma related psychiatric disorders such as PTSD, anxiety disorders secondary to trauma, substance use disorders secondary to trauma, eating disorders secondary to trauma, and depression secondary to prior trauma.

### Definitions

To aid in understanding the detailed description of the compositions and methods according to the disclosure, a few express definitions are provided to facilitate an unambiguous disclosure of the various aspects of the disclosure. Unless defined otherwise, all technical and scientific terms used herein have the meaning commonly understood by a person skilled in the art to which this invention belongs.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The term “consisting essentially of” is meant to encompass the specified materials or steps that do not materially affect the basic and novel characteristic(s)” of the claimed invention.

For example the phrase “consisting essentially of 0.5% by weight” is intended to mean that an excess of 0.5% by weight would materially alter the basic and novel properties of the invention.

The phrases “in one embodiment,” “in various embodiments,” “in some embodiments,” and the like are used repeatedly. Such phrases do not necessarily refer to the same embodiment, but they may unless the context dictates otherwise.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

The terms “activate,” “increased,” “increase” or “enhance” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

The term “therapeutic agent” is used herein to denote a chemical compound, a substance, an active ingredient, or the like that may render it suitable as a “therapeutic agent,” which is a biologically, physiologically, or pharmacologically active substance (or substances) that acts locally or systemically in a subject intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art.

As used herein, “endophenotype” is a quantitative biological trait that in a reproducible, measurable fashion reflects the function (and behavioral endpoint) of a defined biological system and is reasonably heritable, and therefore is more closely related to the root cause of the disease than the broad clinical phenotype. To be considered an endophenotype, the biomarker must fulfill four criteria: (1) it is associated with illness in the population; (2) it is heritable; (3) it is largely state independent (manifests in the individual whether or not the affected individual is symptomatic); and (4) within families, endophenotype and illness cosegregate.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein “patient,” “subject,” “individual,” may be used interchangeably herein.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, absorption delaying agents, and the like that are compatible with the activity of one or more components of the invention, and are physiologically acceptable to the subject. Supplementary active compounds may also be incorporated into the compositions.

“Pharmaceutically acceptable salts” or “pharmaceutically acceptable complexes” refers to salts or complexes of an active ingredient that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and polygalacturonic acid.

The term “derivative” as used herein refers to a chemical substance related structurally to another, i.e., an “original” substance, which can be referred to as a “parent” compound. A “derivative” can be made from the structurally related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing directly or indirectly, the activity disclosed herein.

The terms “sample” and “biological sample” as used herein generally refer to a biological material being tested for and/or suspected of containing an analyte of interest such as antibodies.

As used herein, “treatment” or “treating,” or “palliating” or “ameliorating” are used interchangeably. These terms refer to an approach for obtaining beneficial or desired results, including, but not limited to, a therapeutic benefit and/or a prophylactic benefit. By therapeutic benefit is meant any therapeutically relevant improvement in or effect on one or more diseases (e.g., inflammatory diseases, neurodegenerative diseases, and cardiovascular diseases), conditions, or symptoms under treatment. For prophylactic benefit, the agent or the compositions thereof may be administered to a subject at risk of developing a particular disease, condition, or symptom, or to a subject reporting one or more of the physiological symptoms of a disease, even though the disease, condition, or symptom may not have yet been manifested.

The terms “inhibit,” “decrease,” “reduced,” “reduction,” or “decrease” are all used herein generally to mean a decrease by a statistically significant amount. However, for avoidance of doubt, “reduced,” “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

All methods described herein are performed in any suitable order unless otherwise indicated herein or otherwise clearly contradicted by context. About any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

As used herein, the terms “sign” and “signs” can refer to objective findings of a disorder. For example, a sign may include heart rate and rhythm, body temperature, pattern and rate of respiration, papillary changes and blood pressure. In other instances, signs can be associated with, or indicative of, symptoms.

As used herein, the terms “symptom” and “symptoms” can refer to subjective indications that characterize a disorder. For example, recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response.

As used herein, the term “symptom cluster” can refer to a set of signs, symptoms, or a set of signs and symptoms that are grouped together because of their relationship to each other or their simultaneous occurrence. For example, diagnosis of PTSD may hinge on identifying three symptom clusters: re-experiencing/intrusion; avoidance/numbing; and hyperarousal.

As used herein, the term “re-experiencing/intrusion” can refer to at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders. In some instances, the physiological reactivity can manifest in at least one of abnormal respiration, abnormal cardiac rate of rhythm, abnormal blood pressure, abnormal function of a special sense, and abnormal function of sensory organ.

As used herein, the term “avoidance/numbing” can refer to at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, and sense of a foreshortened future. Restricted range of effect characterized by diminished or restricted range or intensity of feelings or display of feelings can occur. A sense of a foreshortened future can manifest in thinking that one will not have a career, marriage, children, or a normal life span. Avoidance/numbing can also manifest in social anxiety and anxiety with unfamiliar surroundings.

As used herein, the term “hyperarousal” can refer to at least one of difficulty falling or staying asleep, irritability or outbursts of anger, difficulty concentrating, hypervigilance, and exaggerated startle response.

Each publication, patent application, patent, and other reference cited herein is incorporated by reference in its entirety to the extent that it is not inconsistent with the present disclosure. Nothing herein is to be construed as an admission that the present invention is not entitled to antedate such publication by virtue of prior invention.

It is understood that the examples and embodiments described herein are for illustrative purposes only and that various modifications or changes in light thereof will be suggested to persons skilled in the art and are to be included within the spirit and purview of this application and scope of the appended claims

### Trauma-Related Psychopathology

As used, herein trauma related psychiatric disorders are those conditions that are secondary to a prior or an earlier exposure to a traumatic or stressful event, namely exposure to actual or threatened death, serious injury or sexual violence. The new Diagnostic and Statistical Manual of Mental Disorders, fifth edition (DSM-5) set forth an increased recognition of trauma as a precipitant, proposing a commonality in the etiology of all such disorders. To that end, the role of trauma is becoming more apparent in pathophysiology of number of conditions, including but not limited to PTSD, depressive disorders, anxiety disorders, dissociative disorders, neurodevelopmental disorders and other conditions.

It has been suggested that approximately 25% of people exposed to a traumatic event (as defined in criterion A in DSM-5: exposure to death, threatened death, actual or threatened serious injury, actual or threatened sexual violence) will develop PTSD symptoms and 25% of those will go on to demonstrate chronic PTSD. Historically symptoms including shell shock, battle fatigue, accident neurosis and posttraumatic neurosis were used to describe patients who experience psychiatric disorders subsequent to a traumatic experience. However, newer techniques include administration of PTSD diagnostic interviews that can assess the status and symptom severity of patients at risk of developing PTSD.

PTSD is currently diagnosed using the DSM-5, PTSD symptoms are ascertained using the CAPS5. However, other methodologies can be used in measuring at least one sign, symptom, or symptom cluster of PTSD in a patient, also referred in the art as “scale.” In some instances, a scale may be an interview or a questionnaire. Non-limiting examples of scales include Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

It is at least one aspect of the present invention to use any of such scales in combination with an endophenotype to diagnose a subject suffering or at risk of developing, a trauma related psychiatric disorder and further monitor subjects in need of a treatment. Since the interaction between an underlying predisposition in certain individuals and a traumatic event can play a substantial role in improving the sign and symptoms associated with trauma related psychiatric disorders, it can ultimately achieve optimal clinical outcomes for patients suffering from such disorders. As such, it is another aspect of the present invention to employ a cluster of such sign and symptoms using an industry accepted sale in combination with a biomarker to improve patient's clinical outcomes as compared to those not employing such cluster and biomarker parameter.

In addition, trauma is associated with increased risk of developing MDD, substance use disorders, eating disorders, and GDD. These disorders are highly comorbid and share common underlying dimensions of threat- and loss-related symptomatology. The related symptomatology includes re-experiencing symptoms such as intrusive memories and nightmares about a trauma; avoidance of trauma-related reminders; and hyperarousal symptoms such as sleep disturbance and hypervigilance, while loss-related symptomatology includes emotional numbing symptoms such as diminished interest in activities, restricted range of affect, and detachment, as well as dysphoric apathy and generalized anxiety symptomatology. At least in one aspect of the present invention, patients suffering from trauma related MDD, substance use disorders, eating disorders or GDD will undergo the baseline evaluation of such symptoms before initiating the pharmacological treatment contemplated herein.

The phenotypic expression of these threat- and loss-related symptomatology dimensions is best conceptualized as transdiagnostic and dimensional in nature, spanning the full spectrum of symptom severity ranging from no symptoms to severe symptoms. At least one aspect of the present invention is to employ optimal characterization of the phenotypic expression of trauma-related psychopathology in factor analytic and taxometric studies as endpoints in clinical trials of pharmacotherapies directed for more precise and individualized treatment of trauma-related psychopathology.

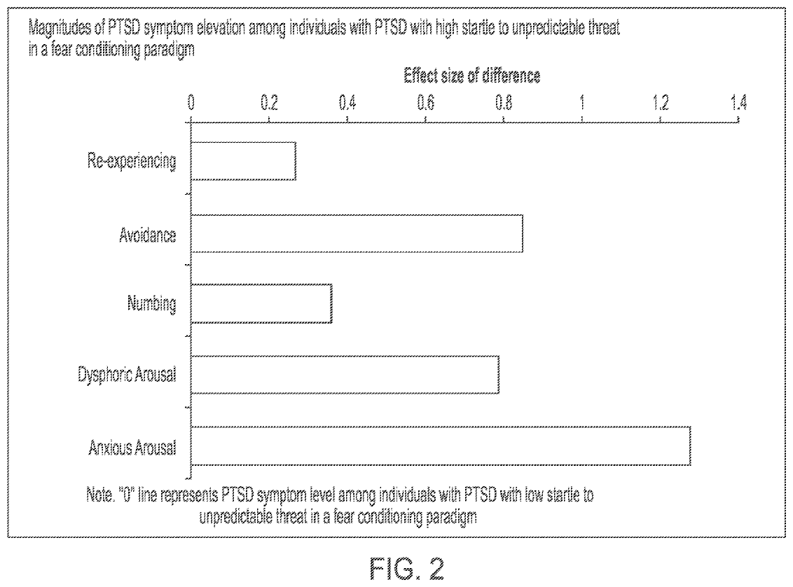

Data from individuals suffering from a diagnosis of PTSD has shown the relationship between measures of startle response to unpredictable threat in a fear-potentiated startle paradigm and the phenotypic expression of PTSD symptomatology using a novel 5-factor model of this disorder. (See FIG. 2) Patients with high startle amplitude to unpredictable threat had highly elevated anxious arousal symptom. Moreover, it appears that the magnitudes of differences in other PTSD symptom clusters were less pronounced (all p's>0.51; all d's<0.85). (See FIG. 2).

In some embodiments, the present invention employs specific endophenotypes that are prevalent in functional characterization of genes associated with the risk of patients suffering from a trauma related psychiatric disorder. Such specific endophenotypes can be used as a tool to identify the risk of patients that have higher degree of predisposition to developing a trauma related psychiatric disorder at some point of their life. In at least one embodiment, the present invention provides a method of treating patients with predisposition to exhibiting a response with high startle amplitude to unpredictable threat had highly elevated anxious arousal symptoms.

In at least another embodiment, the use of the presently disclosed azetidine derivative provides a long-term management of severe anxious arousal symptoms in a subpopulation of posttraumatic patients who display a stronger magnitude association between startle amplitude to unpredictable threat. In at least some embodiments, the present methods further include the identification of a subpopulation of patients who display a functional endophenotypic biomarker. Such endophenotypic biomarker include high startle amplitude to unpredictable threat, degree of the severity of anxious arousal symptoms to such treats or any combination thereof.

In some embodiments, the method of treatment of patients suffering from a trauma-related may further be personalized for the subpopulation in need by determining and monitoring such endophenotypes biomarkers including variations in certain gene expressions, such as variation in the cannabinoid receptor type 1 (CNRI) gene, with the A allele of rs1049353, association of CNR1 SNP rs1049353, childhood physical abuse, or any combinations thereof. Other biomarkers include but are not limited to a genetic variant encoding the function of fatty acid amid hydrolase (FAAH) patients at risk for identifying those subjects carrying a variant allele of the FAAH gene which results in reduced FAAH activity, demonstrating reduced amygdala response to negative stimuli, compared to wild type FAAH allele carriers, thereby providing additional beneficial effect for reducing FAAH activity.

In some embodiments, the endophenotype may include biomarkers such as plasma anandamide levels, plasma cortisol levels, brain CB1 receptor availability, each of which alone or in combination may provide a predictive diagnosis of a trauma related psychiatric disorder with at least 60%, 70%, 80%, 85%, 90%, 95%, 98% or 99% degree of accuracy.

In certain embodiment, neuroimaging studies combined with clinical presentation may be the basis of identifying predisposition to developing a trauma related psychiatric disorder. In some embodiments, employing the same parameter during the diagnosing process such as identification of cluster parameters may become the basis of monitoring and assessing clinical effectiveness of a therapeutic regimen, wherein such endophenotype can link the traumatic event to the disease and patient's phenotypic experience throughout the course of the disorder. In other embodiments, the present invention is directed to identification of such patients and subsequently treating such patients with effective amounts of a therapeutic agent that reverses the disorder, followed by monitoring patient specific cluster parameters and endophenotype profile.

In at least one embodiment, the trauma related psychiatric disorders may be reproduced by acceptable mechanisms or stimulation of specific neural structures. Specifically, threat-related symptomatology includes re-experiencing symptoms such as intrusive memories and nightmares about a trauma; avoidance of trauma-related reminders; and hyperarousal symptoms such as sleep disturbance and hypervigilance, while loss-related symptomatology includes emotional numbing symptoms such as diminished interest in activities, restricted range of affect, and detachment, as well as dysphoric apathy and generalized anxiety symptomatology.

In some embodiments, anxiety disorders, depression, eating disorders or substance use disorders secondary to trauma may be identified through stimulation of a specific neural structure, the amygdala; and in other embodiments, such conditions may be identified by stimulating a predetermined area of a target amygdala that exhibits altered activity relative to its counterpart.

In some embodiments of the present invention, the negative valence systems characterized by heterogeneous clusters of symptoms that are transdiagnostic in nature; span the severity spectrum, may be explored as a parameter to monitor patients responsiveness to the treatments instantly contemplated in the present invention. For example, anxious arousal symptoms, such as exaggerated startle response during fear learning, in particular, have been found to contribute to maintenance of anxiety symptoms in symptomatic trauma survivors.

Hypervigilance to threatening information contributes to and maintains the persistence of trauma-related symptomatology, even months to years after trauma exposure. Greater hyperarousal symptomatology is also associated with exaggerated fear expression and higher overall PTSD symptom severity and impaired extinction in individuals with PTSD. Each of such symptom clusters may be the basis of monitoring the responsiveness of a trauma related therapeutic regimen.

### The Role of Cannabinoid System in Trauma Related Psychiatric Disorders

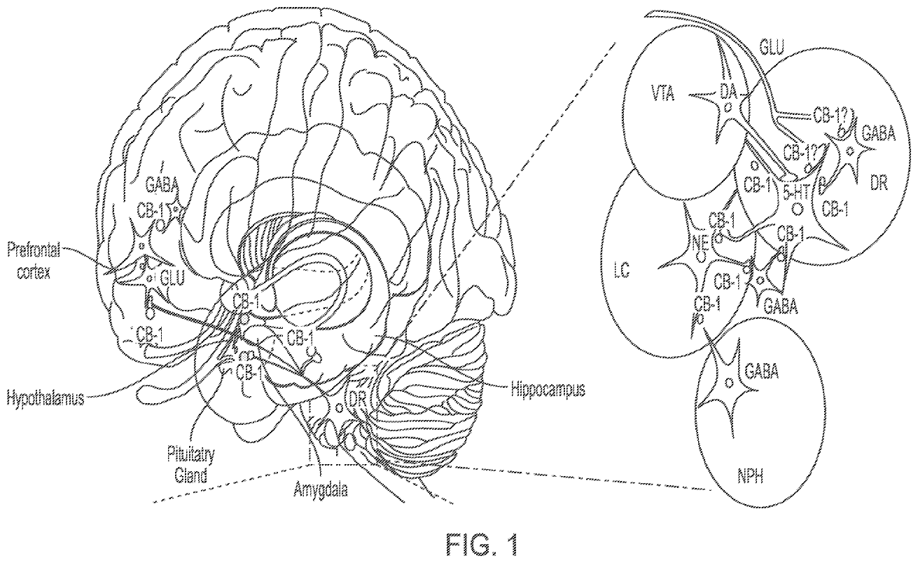

At least one aspect of the present invention is directed to the inventor's realization of the important role of the endocannabinoid (eCB) system in the brain of people with suffering from a trauma related psychiatric disease such as PTSD and the important role of the eCB system in the development of trauma related psychiatric disorders such as PTSD symptoms. Hill et al., 2009 illustrated the brain circuitry that is routinely involved in PTSD. It is now believed that the prefrontal cortex, amygdala and hippocampus form a key tri-nodal circuit to regulate emotion, mood and anxiety. (See FIG. 1) CB1 receptors are found in moderate to high levels throughout these regions. (See FIG. 1)

In some embodiments, cannabinoid anandamide acting via the CB1 receptor provide an stress modulating response by regulating the chronic trauma-mediated effects on brain and body functions. Fatty acid amide anandamide (AEA) is an agonist of the cannabinoid receptors CB1 and CB2, an integral part of the eCB system. AEA is hydrolyzed to arachidonic acid by fatty acid amide hydrolase (FAAH), which is a membrane bound enzyme that appears to act as a critical mediator of the body's own cannabinoid function and thus offering a potential target for novel pharmacotherapy. However, this is the first time that the role of eCB system is being directly linked to the development and prognosis of trauma related psychiatric disorders such as PTSD, MDD and GDD. In some embodiments, elevated CB1 receptors are directly linked to the risk of developing PTSD.

In at least one aspect of the present invention, the eCB system is modulated by increasing the plasma concentration of its endogenous agonists to CB1 and CB2. In some embodiments, the present invention is directed to increasing the plasma AEA level in patients suffering from a trauma related disorder such. In other embodiments, the present invention is directed to increasing the plasma level of other endogenous endocannabinoids such as palmitoylethanolamide (PEA) or oleoylethanolamide (OEA). In some embodiment, the present invention is directed to methods of administering effective therapeutic doses of the azetidine compounds of the present invention to modulate the activity of CB1 receptors of the amygdala region embedded in the PTSD circuitry.

### Methods of Treatment

In at least some embodiments, the present invention is directed to methods of administering to a patient suffering from a trauma related psychiatric disorder a FAAH inhibitor which is a azetidine derivative such as 3-(biphenyl-4-yloxy)-azetidine-1-carboxylic acid phenylamide; 3-(Biphenyl-4-yloxy)-azetidine-1-carboxylic acid (3-fluoro-phenyl)-amide; 3-(Biphenyl-4-yloxy)-azetidine-1-carboxylic acid pyridin-3-ylamide; 3-{5-[3-(2-Methoxy-ethoxy)-phenyl]-pyridin-2-yloxy}-azetidine-1-carboxylic acid pyrimidin-4-ylamide; 3-{5-[3-(2-Methoxy-ethoxy)-phenyl]-pyridin-2-yl oxy}-azetidine-1-carboxylic acid pyrazin-2-ylamide; 3-{5-[3-(2-Methoxy-ethoxy)-phenyl]-pyridin-2-yloxy}-azetidine-1-carboxylic acid pyridazin-3-ylamide; 3-[5-(2-Fluoro-phenyl]-pyridin-2-yloxy]-azetidine-1-carboxylic acid pyrazin-2-ylamide; 3-[5-(2-Fluoro-3-methoxy-phenyl)-pyridin-2-yloxy]-azetidine-1-carboxylic acid pyridazin-3-ylamide; 3-[5-(2-Fluoro-phenyl)-pyridin-2-yloxy]-azetidine-1-carboxylic acid (6-methoxy-pyridazin-3-yl)-amide; 3-{[5-(2-Fluorophenyl)pyridin-2-yl]oxy}-N-(pyridazin-3-yl)azetidine-1-carboxamide or a pharmaceutically acceptable salt thereof described in the U.S. Pat. No. 8,450,346 incorporated herein in its entirety.

In at least one embodiment, the present invention is directed to methods of treating patient suffering from a trauma related psychiatric disorder such as post-traumatic anxiety disorder, post-traumatic depression, MDD, GDD and any combinations thereof. Although PTSD is often viewed as a unitary, categorical diagnostic entity, the symptom presentation in PTSD reflects negative valence systems implicated in trauma-related psychopathology (i.e., threat and loss-related symptomatology) are often considered to be homogeneous in nature and representative of a general construct of psychological distress.

The use of FAAH inhibitors has previously been described for treatment of pain. For example, the U.S. Pat. Nos. 8,450,346; 9,006,269; 9,475,800 and 10,383,871 all of which are incorporated herein in its entirety describes various azatidine derivatives, their methods of manufacturing and using them for treatment of pain. At least one mode of action for the respective FAAH inhibitors includes increasing plasma levels of endogenous CB1 and CB2 receptor agonists. For the first time, the present inventors employ such compounds in treating psychiatric disorders that are secondary to trauma.

As such at least one aspect of the present invention employing active ingredients that can target and modulate the activity of the eCB system in patients suffering from trauma induced psychiatric conditions. Without wishing to be bound by any particular theory, it is suggested that at least one approach to treatment of post-trauma psychiatric stress disorders is identifying the critical biological and behavioral markers of chronic stress and fear to the eCB system. In some embodiments, subjects at risk of developing stress induced psychiatric conditions are monitored for their genetic and phenotypic dispositions at the initial phases of the treatment.

In some embodiments suitable parameters are identified as the key diagnostic and/or monitoring components for predicting the responsiveness to a medical treatment including administration of an azetidine derivative namely 3-[5-(2-Fluoro-phenyl)-pyridin-2-yloxy]-azetidine-1-carboxylic acid (6-methoxy-pyridazin-3-yl)-amide; 3-{[5-(2-Fluorophenyl)pyridin-2-yl]oxy}-N-(pyridazin-3-yl)azetidine-1-carboxamide.

In at least some embodiments, subjects suffering or at risk of developing a post-trauma psychiatric stress disorder such as PTSD may benefit from receiving pharmacologically effective doses of azetidine derivatives that are both effective and safe. In at least some embodiments, subjects suffering from a post-trauma psychiatric stress disorder included those with suffering from hyperactive arousal symptoms. In at least one embodiment, the patient may be suffering from PTSD with predominant anxious arousal symptoms.

In some embodiments, the azetidine derivative 3-{[5-(2-Fluorophenyl)pyridin-2-yl]oxy}-N-(pyridazin-3-yl)azetidine-1-carboxamide or a pharmaceutically acceptable salt thereof is administered at therapeutically effective amounts to subjects suffering from a post-trauma psychiatric stress disorder to restore therapeutic anandamide levels, and reduce fatty acid amide hydrolase (FAAH) activity after severe trauma. In other embodiments, the azetidine derivative inhibits pathological amygdala hyperreactivity and facilitates activating and function at the prefrontal cortex of the subject's brain, thereby facilitating the more rapid habituation to threat.

In certain embodiments, the administration of the therapeutically effective amounts of an inhibitor of FAAH, restores the PTSD-characteristic hypothalamic-pituitary-adrenal (HPA)-axis dysregulation, facilitating improvements of at least one sign or symptom associated with hyperarousal activity. In other embodiments, the administration of the therapeutically effective amounts of an inhibitor of FAAH promotes sleep and rapid eye movement (REM) sleep, which can reduce incidences of re-experiencing and hyperactive consolidation of traumatic memories during sleep.

In some embodiments, the administration of the therapeutically effective amounts of an inhibitor of FAAH in subjects in need reduces sympathetic tone via activation of CB1 receptors on noradrenergic nerve terminals. In other embodiment, such method is able to modulate other cannabinoids, which the body produces, such as palmitoylethanolamide (PEA) or oleoylethanolamide (OEA), which are anti-inflammatory and analgesic, and regulate satiety, respectively. In addition to FAAH inhibitors having the potential to reduce PTSD symptoms, they may also help mitigate altered pain sensitivity, as well as low-grade inflammation.

In at least another embodiment, the bio-mechanism-based interventions that target anxious arousal symptoms of PTSD may help to alleviate more deleterious downstream effects of this disorder, such as re-experiencing and emotional numbing symptoms or suicidality. Accordingly, blocking FAAH, the enzyme that degrades endogenous anandamide, and thereby increases levels of anandamide. These effects can provide an early pharmacotherapeutic intervention that can mitigate the more deleterious downstream effects of anxious arousal symptoms in patients with PTSD. As such in at least one embodiment, the present invention is directed to increasing the plasma levels of anandamide in such patients suffering from a trauma related psychiatric disorder such as PTSD, MDD, substance use disorders, eating disorders, GDD, or any combinations thereof.

In some embodiments, the inventors employ specific endophenotypes that are prevalent in functional characterization of risk genes associated with stress related genetic predisposition can be used as a monitoring tool for those patients diagnosed with PTSD who are candidate to receive a therapeutic regimen of the azetidine derivative 3-{[5-(2-Fluorophenyl)pyridin-2-yl]oxy}-N-(pyridazin-3-yl)azetidine-1-carboxamide or a pharmaceutically acceptable salt thereof.

In other embodiments, the present invention highlights a number of putative endophenotypes for trauma related psychiatric disorder that can be used as a tool to monitor the effectiveness of a FAAH inhibitor treatment. Other monitoring parameters may include symptoms such as learning impairments, reduced reward functioning, increased stress sensitivity, REM sleep abnormalities, functional and structural brain abnormalities, dysfunctions in neurotransmitter activities, and intracellular signal transaction measures.

In some embodiments, the present inventors employ a comprehensive, multimodal assessment approach that will capture the broad range of manifestations of anxious arousal. This assessment approach will include a combination of clinical interviews, functional endophenotypic measures of anxious arousal, including a functional magnetic resonance imaging (fMRI) fear-potentiated startle task, which provide objective measures of the core components of anxious arousal-hypervigilance and exaggerated startle response.

In other embodiments, the invention is directed to identifying the internal phenotypes that lie on the pathway between patient's genetic profile and the risk of developing a stress related psychiatric disorders. In some embodiments, the present invention includes phenotype. In some embodiments, those at risk of developing a stress related psychiatric disease exhibit a higher frequency of genetic profiles associated with developing such conditions as compared to those in the general population. In one embodiment, the presence of such genetic profiles is heritable tend to co-segregate with the illness in multiple affected families.

In some embodiments, the FAAH enzyme inhibitor is 3-{[5-(2-Fluorophenyl)pyridin-2-yl]oxy}-N-(pyridazin-3-yl)azetidine-1-carboxamide or a pharmaceutically acceptable salt thereof to treat a trauma related disorder in population specifically when anxious arousal symptoms are present. In some embodiments, the FAAH inhibitor or a pharmaceutically acceptable salt thereof, or pharmaceutically acceptable prodrug thereof, is administered prophylactically.

In some embodiments, the inhibitor, a pharmaceutically acceptable salt thereof, a pharmaceutically active derivative thereof or pharmaceutically acceptable prodrug thereof is administered intratumorally, intravenously, subcutaneously, intraosseously, orally, transdermally, in sustained release, in controlled release, in delayed release, as a suppository, or sublingually.

In at least one embodiment, the present invention is directed to methods of treating patient suffering from a trauma related psychiatric disorder such as post-traumatic anxiety disorder, post-traumatic depression, post-traumatic eating disorders and substance use disorders, GDD and any combinations thereof. Although PTSD is often viewed as a unitary, categorical diagnostic entity, the symptom presentation in PTSD reflects negative valence systems implicated in trauma-related psychopathology (i.e., threat and loss-related symptomatology) are often considered to be homogeneous in nature and representative of a general construct of psychological distress.

In this new approach to PTSD treatment development links critical biological and behavioral markers of chronic stress and fear to the emerging evidence for an important role of the eCB system in the etiology of stress and fear-related disorders including PTSD. This concept therefore provides for the next generation PTSD treatments that can be developed that are both effective and safe.

In some embodiments, the course of treatment ranges from at least 2 weeks to 12 month, on a daily regimen.

The invention further provides pharmaceutical formulations which include therapeutically effective amounts of the azetidine derivative 3-{[5-(2-Fluorophenyl)pyridin-2-yl]oxy}-N-(pyridazin-3-yl)azetidine-1-carboxamide or a pharmaceutically acceptable salt thereof, pharmaceutically acceptable solvates, or pharmaceutically acceptable salts solvated with pharmaceutically acceptable solvents thereof, and one or more pharmaceutically acceptable carriers, diluents, or excipients.

The carrier(s), diluent(s) or excipient(s) must be acceptable in the sense of being compatible with the other ingredients of the formulation, capable of pharmaceutical formulation, and not deleterious to the recipient thereof. The invention also provides a process for the preparation of a pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient or the pharmaceutical formulations may be presented in unit dose forms containing a predetermined amount of active ingredient per unit dose. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

In another aspect, new combinations and compositions containing at least one active ingredient that can provide supportive or additive therapeutic benefits. Such pharmacologically active ingredients may include ketamine, antidepressants: e.g., biogenic amine non-selective reuptake inhibitors, e.g., tricyclic antidepressants like imipramine; serotonin selective reuptake inhibitors like fluoxetine (Prozac); monoamine oxidase inhibitors (MAO-I) like phenelezine; other types of antidepressant medications including atypical antidepressants. Antidepressants augmentation with other medications e.g., lithium, T3, T4, etc. Other treatment modalities with antidepressant effects: electro-convulsive treatment (ECT); light therapy, psychotherapy e.g., cognitive or interpersonal therapy for PTSD. More preferably, the FAAH inhibitor of the present invention may be combined with therapeutic agent that is selected from the group consisting of a serotonin reuptake inhibitors (SSRIs), a beta-adrenergic antagonist, an alpha 1 adrenergic antagonist, a benzodiazepine, an opiate compound, a cannabinoid compound, a cortisol lowering agent, an antagonist of N-methyl-D-aspartate (NMDA) receptors, a ventromedial prefrontal cortex enhancer and any combinations thereof.

Other active ingredients that can ameliorate symptoms of a neuropsychiatric disorder, include but are not limited to antidepressants such as lithium salts, carbamazepine, valproic acid, p-chlorophenylalanine, p-propyidopacetamide dithiocarbamate derivatives e.g., FLA 63; anti-anxiety drugs, e.g., diazepam; monoamine oxidase (MAO) inhibitors, e.g., iproniazid, clorgyline, phenelzine, tranylcypromine, and isocarboxazid; biogenic amine uptake blockers, e.g., tricyclic antidepressants such as desipramine, imipramine and amitriptyline; atypical antidepressants such as mirtazapine, nefazodone, bupropion; serotonin reuptake inhibitors e.g., fluoxetine, venlafaxine, and duloxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), S and dibenzodiazepines (e.g., clozapine); benzodiazepines; dopaminergic agonists and antagonists e.g., L-DOPA, cocaine, amphetamine, a-methyl-tyrosine, reserpine, tetrabenazine, benztropine, pargyline; noradrenergic agonists and antagonists e.g., clonidine, phenoxybenzamine, phentolamine, tropolone.

As such the combinations of the present invention can be administered in such oral dosage forms as tablets, capsules (each of which includes sustained release or timed release formulations), pills, powders, micronized compositions, granules, elixirs, tinctures, suspensions, syrups and emulsions. Likewise, they may be administered in intravenous (bolus or infusion), subcutaneous, intramuscular or transdermal (e.g., patch) forms. The ordinarily skilled physician, veterinarian or clinician can readily determine and prescribe the effective amount of the present combination required to prevent, counter or arrest the progress of the urinary retention associated conditions.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 50, 100, 150, 200, 250, 300, 350, 400, 425, 450, 500, 525, 550 and 600 milligrams of the respective active ingredient for the symptomatic adjustment of the dosage to the patient to be treated.

The components of the presently describe oral formulations are conventional pharmaceutical carriers known formulations for oral use wherein said components are mixed together or separated, for example in two tablets introduced in a capsule or in a two-compartment capsule or in a multilayer (di-layer) tablet wherein the two components are both in IR or in ER form or one of the two components is in IR form and the other is in ER form, according to known technologies. In one embodiment, the compositions of the present may be in immediate, sustained or extended release pharmaceutical formulations. For example, the present invention includes compositions that comprise from 0 to 50% of an immediate release component including a FAAH inhibitor or a combination thereof and up to 100% of an extended release particle, which comprises the same or different type active ingredient.

Additionally, the FAAH inhibitors according to the present invention can be delivered in microparticles composed of various biocompatible, biodegradable polymers. Examples of these types of polymers include polyester, polyalkylcyanoacrylate, polyorthoester, polyanhydride, albumin, gelatin, and starch. An advantage of microparticles is that they provide controlled and sustained release of the agent thereby minimizing the required dosing frequency. Therefore, compositions of the present invention may contain plurality of microparticles both in immediate release or delayed forms.

The combination therapies according to the present invention include the administration of at least one of the azatiedine derivatives described herein and any one of the active ingredients described above. Such combination of agents may be administered together or separately and, when administered separately this may occur simultaneously or sequentially in any order, both close and remote in time.

Pharmaceutical formulations adapted for oral administration may be presented as discrete units such as capsules or tablets; powders or granules; solutions or suspensions in aqueous or non-aqueous liquids; edible foams; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be used in granulating. The powder mixture can be run through a tablet machine, and if the result is imperfectly formed slugs, they can be broken into granules, and the granules can be lubricated and incorporated back into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like.

Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant, disintegrant, and pressing into tablets. A powder mixture is prepared by mixing the compound, suitably comminuted, with a diluent or base as described above. The powder mixture can be granulated by wetting with a binder such as syrup, starch paste, acadia mucilage or solutions of cellulosic or polymeric materials and forcing through a screen. As an alternative to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Where appropriate, dosage unit formulations for oral administration can be microencapsulated. The formulation can also be prepared to prolong or sustain the release as, for example, by coating or embedding particulate material in polymers, waxes or the like.

The compounds may also be coupled with soluble polymers as targetable drug carriers. Such polymers can include, without limitation, polyvinylpyrrolidone, pyran copolymer, polyhydroxypropylmethacrylamide-phenol, poly-ydroxyethylaspart-amide-phenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels for dermal implant delivery. Pharmaceutical formulations adapted for transdermal administration may be presented as discrete patches intended to remain in intimate contact with the epidermis of the recipient for a prolonged period of time.

Pharmaceutical formulations adapted for parenteral administration include aqueous and non-aqueous sterile injection solutions that may contain anti-oxidants, buffers, bacteriostats and solutes that render the formulation isotonic with the blood of the intended recipient; and aqueous and non-aqueous sterile suspensions that may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets.

According to the present invention, azatiedine derivatives of the present invention may be present in amounts of from 1 mg to 1000 mg, advantageously from 10 mg to 750 mg, preferably from 150 mg to 500 mg, per dosage per day. In a preferred embodiment, the azatiedine derivatives FAAH inhibitor is 3-{[5-(2-Fluorophenyl)pyridin-2-yl]oxy}-N-(pyridazin-3-yl)azetidine-1-carboxamide or a pharmaceutically acceptable salt thereof. In at least one embodiment, 3-{[5-(2-Fluorophenyl)pyridin-2-yl]oxy}-N-(pyridazin-3-yl)azetidine-1-carboxamide may be given to subjects suffering from PTSD at doses amounting to about 50 mg to about 750 mg or preferably at about 100 mg to about 500 mg per day.

In one embodiment, the presently described combination regimen improves the hyperexitability by reducing it to at least 50%, 75%, 85%, 90%, 95%, and 98% as compared to the pretreatment measurements of any sign or symptoms.

In other embodiments, the present invention is directed to methods of restoring back to normal low levels of anandamide-caused by increasing FAAH activity after severe trauma-which represent a vulnerability factor to developing PTSD including administering to a patient suffering from such condition a daily dose of 100 to 500 mg per day of 3-{[5-(2-Fluorophenyl)pyridin-2-yl]oxy}-N-(pyridazin-3-yl)azetidine-1-carboxamide or a pharmaceutically acceptable salt thereof.

In some embodiments, the present invention is directed to methods of suppressing amygdala hyperreactivity and activating prefrontal cortex functions, thereby facilitating the more rapid habituation to threat storing the PTSD-characteristic hypothalamic-pituitary-adrenal (HPA)-axis dysregulation. In yet another embodiment, the present invention is directed to method of promoting sleep and suppressing of rapid eye movement (REM) sleep, which can increase re-experiencing and hyperconsolidation of traumatic memories during sleep. In yet another embodiment, the method is directed to reducing sympathetic tone via activation of CB1 receptors on noradrenergic nerve terminals and Modulating other cannabinoids which the body produces, such as palmitoylethanolamide (PEA) or oleoylethanolamide (OEA), which are anti-inflammatory and analgesic, and regulate satiety, respectively. In addition to FAAH inhibitors having the potential to reduce PTSD symptoms, they may also help mitigate altered pain sensitivity, as well as low-grade inflammation.

In at least one embodiment, the FAAH of choice is 3-{[5-(2-Fluorophenyl)pyridin-2-yl]oxy}-N-(pyridazin-3-yl)azetidine-1-carboxamide or a pharmaceutically acceptable salt thereof, administered at doses ranging from about 150 mg/day to about 750 mg/day. In some embodiments, optimal dosing achieves serum C_{trough} levels ranging from about 100 ng/ml to about 700 ng/ml for optimal clinical benefits. In yet another embodiment, the same is administered at doses of about 300 mg/day to about 500 mg/day to achieve a mean C_{trough} estimate ranging from about 200 ng/ml to about 600 ng/ml.

In certain embodiments, 3-{[5-(2-Fluorophenyl)pyridin-2-yl]oxy}-N-(pyridazin-3-yl)azetidine-1-carboxamide will be at steady state after 5, 6, 7, 8, 9 or 10 days administration of a dose ranging from about 150 mg/day to about 750 mg/day. In certain scenarios, if the azethidine derivative is administered at a similar time on days 1 through 10, or preferably 3 through 8 (and previous days), the pre-scan plasma will effectively be the trough steady-state level (C_{trough}) from the dose administered on day 7. In such scenario, the estimated plasma concentration of can fall within in the range of about 200 ng/ml to about 600 ng/ml.

In other embodiments, upon achieving trough steady state plasma, plasma FAA levels will be minimal. Conversely, at the 450 mg/day dose, changes in plasma FAA levels in response to 3-{[5-(2-Fluorophenyl)pyridin-2-yl]oxy}-N-(pyridazin-3-yl)azetidine-1-carboxamide ranging from 200 nm/ml to about 600 ng/ml are predicted to be maximal; in the order of 3, 4, 8 and 5 ng/mL for AEA, LEA, OEA and PEA, respectively.

In some embodiment, the patients undergoing FAAH inhibitor regimen will exhibit functional magnetic resonance imaging (fMRI) measure of resting state fMRI, physiological responses a standard fear extinction paradigm, and clinician- and self-report measures of trauma-related psychopathology with focus on reductions in composites scores of anxious arousal symptoms and a reduction in CAPS-5 total score at day 8 relative to baseline. In other embodiments, exploratory endpoints will also include changes in psychosocial functioning (QoL), sleep, activity, measures of plasma endocannabinoids, vegetative symptoms, FAAH activity in leukocytes and allele and genotype frequency of the FAAH C385A locus (rs324420) and CNR1 locus (rs1049353) and their impact on outcome measures.

### EXAMPLES

### Example 1—Establishing the Relationship Between the Phenotype (Hyperarousal) and the Endophenotye Ascertained Using a Fear Conditioning Model

Ten individuals with current PTSD were recruited to evaluate the relation between measures of startle response to unpredictable threat in a fear-conditioning paradigm and the phenotypic expression of PTSD symptomatology using a novel 5-factor model of this disorder. The mean age of this sample was 24.5 (SD=5.3; range=18-38); 6 (60%) were women; 5 (50%) were African-American, 3 (30%) were Caucasian, and 2 (20%) were Hispanic. Mean age of first trauma exposure in this sample was 7.0 (SD=7.5; range=1-18). The most commonly endorsed worst trauma was sexual abuse (n=7; 70%); and the mean duration of time from first trauma exposure to date of study participation was 17.5 years (SD=8.0; range=5-26). The mean total CAPS score was 62.5 (SD=19.7; range=45-88). Due to the non-normal distribution of startle amplitude values, a median split procedure was performed on this variable prior to conducting analyses.

Results of independent-samples t-tests revealed that, compared to individuals with low startle amplitude, those with high startle amplitude to unpredictable threat had highly elevated anxious arousal symptoms (p=0.14; d=1.27; FIG. 2; magnitudes of differences in other PTSD symptom clusters were less pronounced (all p's>0.51; all d's<0.85). Further, Spearman rank correlations revealed a stronger magnitude association between startle amplitude to unpredictable threat and severity of anxious arousal symptoms (r=0.63) compared to other symptom clusters (all r's<0.40). Taken together, these preliminary data provide initial evidence of convergent validity between a functional biomarker-startle amplitude to unpredictable threat—and severity of anxious arousal symptoms.

### Example 2

Exploratory Behavioral Analyses 1:

(a). To evaluate the effect of daily 450 mg of 3-{[5-(2-Fluorophenyl)pyridin-2-yl]oxy}-N-(pyridazin-3-yl)azetidine-1-carboxamide relative to placebo in mitigating PTSD symptoms in adults with PTSD. Patient will be receiving a 450 mg dose of the study agent. According as compared to placebo, those in the treatment arm will be associated with a reduction in overall severity of PTSD symptoms.

(b). To evaluate the effect of 450 mg of the study drug relative to placebo in mitigating PTSD symptoms that comprise a contemporary, five-factor phenotypic model of this disorder, as well as secondary “intermediate phenotype” measures of anxiety sensitivity and somatic anxiety. It is contemplated that, relative to placebo, a 450 mg daily dose of 3-{[5-(2-Fluorophenyl)pyridin-2-yl]oxy}-N-(pyridazin-3-yl)azetidine-1-carboxamide will have the most pronounced effect on reducing anxious arousal (i.e., exaggerated startle, hypervigilance) PTSD symptoms, as well as secondary measures of anxiety sensitivity and somatic anxiety.

### Example 3

Exploratory Behavioral Analyses 2:

(a). Patients at risk of developing PTSD or diagnosed with such condition were evaluated at baseline visit for the differential association between objective measures of startle magnitude in a fear-extinction paradigm, and severity of re-experiencing, avoidance, numbing, dysphoric arousal, and anxious arousal symptomatology from a contemporary, 5-factor phenotypic model of PTSD.

(b). Patients at risk of developing PTSD or diagnosed with such condition were evaluated to determine how 3-{[5-(2-Fluorophenyl)pyridin-2-yl]oxy}-N-(pyridazin-3-yl)azetidine-1-carboxamide exposure-related changes in objective measures of startle magnitude and anxious arousal symptoms relate to changes in re-experiencing, avoidance, numbing, and dysphoric arousal PTSD symptomatology from a contemporary, 5-factor phenotypic model of PTSD. The inventors contemplate that (a) startle magnitude will be most strongly related to severity of anxious arousal symptoms; and (b) the response will be greater for 3-{[5-(2-Fluorophenyl)pyridin-2-yl]oxy}-N-(pyridazin-3-yl)azetidine-1-carboxamide. Accordingly, reductions in startle magnitude and anxious arousal symptoms will be associated with greater reductions in other PTSD symptom clusters.

### Example 4—the Role of the eCB Signaling in Patient Suffering from PTSD

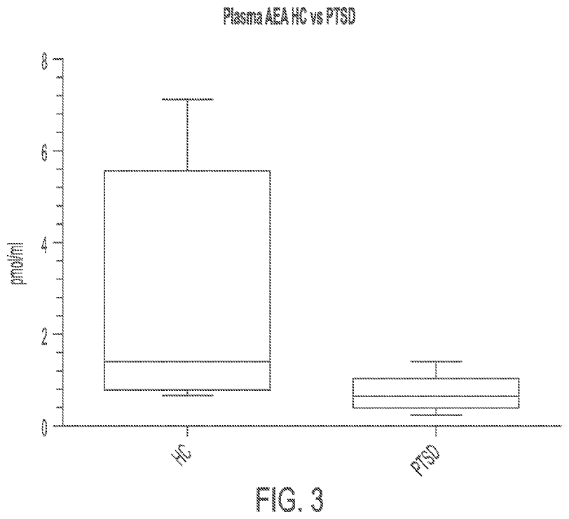

In this study, the inventors evaluated the role of AEA level in patients PTSD patients. Lower eCB levels and corresponding elevations of brain CB1 receptors show a unique linked to PTSD. Accordingly, the Plasma AEA levels were measured in PTSD patients. Results are shown in FIG. 3. It is our observation that plasma AEA levels in PTSD patients are decreased relative to healthy control subjects (0.72±0.12 vs 2.74±0.85 pmol/ml, t=2.47, df=17, p=0.024; FIG. 3). For the first time, the present inventors have been able to provide evidence that altered eCB signaling is observed in PTSD patients. Such initial evidence was later confirmed in multiple independent cohorts of individuals with PTSD.

In addition, earlier age at first trauma was correlated with lower AEA levels in PTSD (r=0.45, p=0.073) and the magnitude of the decrease was associated with the length of illness (r=−0.48, p=0.059). These results support an important role of dysfunctional eCB signaling in PTSD.

While the invention has been described with references to specific embodiments, modifications and variations of the invention may be construed without departing from the scope of the invention, which is defined in the following claims.

What we claim is:

1. A method of treating trauma-related psychiatric disorders in a patient in need thereof comprising the steps of (a) selecting a patient suffering from symptoms associated with said disorder, (b) administering to the patient effective amounts of a fatty acid amide hydrolase (FAAH) inhibitor, (c) identifying an improvement of at least one of the patient's symptoms from the baseline.

2. The method of claim 2, wherein the post-traumatic psychiatric disorder is selected from the group consisting of post-traumatic stress disorder (PTSD), post-traumatic anxiety disorder, post-traumatic depression, post-traumatic substance use disorders, and post-traumatic eating disorders, GDD and any combinations thereof.

3. The method of claim 1, wherein at least one of the symptoms is associated to hyperarousal and exaggerated startle.

4. The method of claim 3, wherein the symptoms associated with hyperarousal is selected from the group consisting of (a) irritable behavior, (b) angry outbursts, (c) recklessness, (d) self-destructive behavior, (e) hypervigilance, (f) exaggerated startle response, (g) problems with concentration, (i) sleep disturbances or any combinations thereof.

5. The method of claim 4, wherein the hyperarousal symptom is manifested for at least one month after the patient's exposure to trauma.

6. The method of claim 1, wherein the trauma is secondary to a fear conditioning model.

7. The method of claim 1, wherein the patient further exhibits abnormal cortisol levels, abnormal endocannabinoid concentrations, a smaller hippocampus than average population measurements, or any combinations thereof.

8. The method of claim 1, wherein the FAAH inhibitor is selected from the group consisting of an azetidine derivative.

9. The method of claim 8, wherein the azetidine derivative is 3-{[5-(2-Fluorophenyl)pyridin-2-yl]oxy}-N-(pyridazin-3-yl)azetidine-1-carboxamide or a pharmaceutically acceptable salt thereof.

10. The method of claim 9, wherein the pharmaceutically acceptable salt is selected from the group consisting of a salt of hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, polygalacturonic acid, and any combinations thereof.

11. The method of claim 10, wherein the azetidine derivative is administered in doses ranges from 1 mg to 1000 mg.

12. The method of claim 1, further including the step of administering to said patient a second therapeutic agent that is selected from the group consisting of a serotonin reuptake inhibitors (SSRIs), a beta-adrenergic antagonist, an alpha 1 adrenergic antagonist, a benzodiazepine, an opiate compound, a cannabinoid compound, a cortisol lowering agent, an antagonist of N-methyl-D-aspartate (NMDA) receptors, a ventromedial prefrontal cortex enhancer and any combinations thereof.

13. A method of treating patients at risk of developing post-traumatic psychiatric disorder comprising the steps of (1) determining the patient's endophenotype, (2) among those identified in step (1), selecting those patients who exhibit at least one of the symptom selected from the group consisting of a) irritable behavior, (b) angry outbursts, (c) recklessness, (d) self-destructive behavior, (e) hypervigilance, (f) exaggerated startle response, (g) problems with concentration, (i) sleep disturbances or any combinations thereof, (3) administering to the patients identified in step (2) an effective amount of an FAAH inhibitor, (4) evaluating the patient's improvement of at least one the symptoms, and (5) continue administering the FAAH inhibitor to those patients who show improvement in at least one of the symptoms.

14. The method of claim 13, wherein the post-traumatic psychiatric disorder is selected from the group consisting of post-traumatic stress disorder (PTSD), post-traumatic anxiety disorder, post-traumatic depression, post-traumatic substance use disorder, and post-traumatic eating disorder.

15. The method of claim 14, wherein at least one of the symptoms is associated to hyperarousal and exaggerated startle.

16. The method of claim 15, wherein the symptoms associated with hyperarousal is selected from the group consisting of (a) irritable behavior, (b) angry outbursts, (c) recklessness, (d) self-destructive behavior, (e) hypervigilance, (f) exaggerated startle response, (g) problems with concentration, (i) sleep disturbances or any combinations thereof.

17. The method of claim 16, wherein the hyperarousal symptom is manifested for at least one month after the patient's exposure to trauma.

18. The method of claim 17, wherein the trauma is secondary to a fear conditioning model.

19. The method of claim 13, wherein the patient further exhibits abnormal cortisol levels, abnormal endocannabinoid concentrations, a smaller hippocampus than average population measurements, or a reduced volume of prefrontal regions of the brain as compared to population measurements, or any combinations thereof.

20. The method of claim 13, wherein the FAAH inhibitor is selected from the group consisting of an azetidine derivative.

21. The method of claim 20, wherein the azetidine derivative is 3-{[5-(2-Fluorophenyl)pyridin-2-yl]oxy}-N-(pyridazin-3-yl)azetidine-1-carboxamide or a pharmaceutically acceptable salt thereof.

22. The method of claim 21, wherein the pharmaceutically acceptable salt is selected from the group consisting a salt of hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, polygalacturonic acid, and any combinations thereof.

23. The method of claim 22, wherein the azetidine derivative is administered in doses ranges from 1 mg to 750 mg.

24. The method of claim 23, further including the step of administering to said patient a second therapeutic agent that is selected from the group consisting of a serotonin reuptake inhibitors (SSRIs), a beta-adrenergic antagonist, an alpha 1 adrenergic antagonist, a benzodiazepine, an opiate compound, a cannabinoid compound, a cortisol lowering agent, an antagonist of N-methyl-D-aspartate (NMDA) receptors, a ventromedial prefrontal cortex enhancer and any combinations thereof.

25. A method of treating anxiety, pain, depression associated with trauma comprising administering to a subject in need of such treatment an effective amount of a FAAH inhibitor.

26. The method of claim 25, wherein the FAAH inhibitor is selected from the group consisting of an azetidine derivative.

27. The method of claim 26, wherein the azetidine derivative is 3-{[5-(2-Fluorophenyl)pyridin-2-yl]oxy}-N-(pyridazin-3-yl)azetidine-1-carboxamide or a pharmaceutically acceptable salt thereof.

28. The method of claim 27, wherein the pharmaceutically acceptable salt is selected from the group consisting of a salt of hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, polygalacturonic acid, and any combinations thereof 30.

29. The method of claim 28, wherein the azetidine derivative is administered in doses ranges from 1 mg to 750 mg.

30. A pharmaceutical unit form which comprises an immediate release and a delayed release component, wherein the immediate release component comprising a first therapeutic agent selected from the group consisting of a FAAH inhibitor, and the delayed release component comprising a second therapeutic agent that is selected from the group consisting of a serotonin reuptake inhibitors (SSRIs), a beta-adrenergic antagonist, an alpha 1 adrenergic antagonist, a benzodiazepine, an opiate compound, a cannabinoid compound, a cortisol lowering agent, an antagonist of N-methyl-D-aspartate (NMDA) receptors, a ventromedial prefrontal cortex enhancer and any combinations thereof.
